(19) United States
(12) Patent Application Publication
(10) Pub. No.: US 2024/0277943 A1
(43) Pub. Date: Aug. 22, 2024

(54) SEQUENCE-DEPENDENT ACTIVATION MECHANISM USING A ROTATOR

(71) Applicant: SHL MEDICAL AG, Zug (CH)

(72) Inventor: Shinshiuan CHIOU, Zug (CH)

(21) Appl. No.: 18/570,885

(22) PCT Filed: Jun. 30, 2022
(86) PCT No.: PCT/EP2022/068020

§ 371 Date: Dec. 15, 2023

(30) Foreign Application Priority Data
Jul. 16, 2021 (EP) 21186182.8

(51) Int. Cl. A61M 5/315

(52) U.S. Cl. CPC A61M 5/31571; A61M 5/31585

(57) ABSTRACT

A medicament delivery device (100) is provided, including a release button (110), a delivery member cover (112), and a rotator (114). The device is operable between a first limiting state (S1) wherein the release button is at most partially pushed and where further pushing is possible but not sufficient to cause a triggering of a delivery of a medicament from the device, a second limiting state in which further pushing of the release button is prevented, and a non-limiting state in which pressing the release button to directly or eventually trigger the delivery of the medicament is possible. The release button, delivery member cover and the rotator are so configured an arranged that in the second limiting state, pressing on the delivery member cover does not cause a transition of the device into the non-limiting state. A corresponding rotator is also provided.

### TECHNICAL FIELD

The present disclosure relates to the field of medicament delivery devices. In particular, the present disclosure relates to such a device having a sequence-dependent activation mechanism using a rotator.

### BACKGROUND

Autoinjectors and other types of medicament delivery devices are designed to allow for a user (e.g. a patient) to perform self-administration of a dose of a particular medicament in a controlled way. For many such devices, the user is normally expected to first position the device against the desired site of injection and to then push a release button of the device in order to trigger a delivery of the medicament from the device. On some devices, pressing or pushing the release button has no effect until the user has first also deactivated one or more safety mechanisms. Such safety mechanisms can for example include having to first push the device against the site of injection, having to remove a safety pin, or similar. When finally pushing the release button, a force element (such as a preloaded spring, or similar) of the device is released and causes a delivery member (such as a needle/syringe) of the device to puncture the injection site, followed by an injection of a dose of medicament through the delivery member and into the body of the user.

In a particular such setup, the safety mechanism includes a delivery member cover and a rotator connected together such that a linear motion of the delivery member cover translates into a rotational motion of the rotator. The rotator is in turn configured such that, by pressing/pushing on the delivery member cover against the injection site, the rotator can be moved from a first alignment relative to the release button wherein it prevents pushing of the release button, to a second alignment relative to the release button wherein it does no longer prevent the pushing of the release button.

However, in for example a stressful situation, or due to lack of earlier experience with the device, the user may not know or at least forget in which order to perform the above tasks. For example, the user may end up pressing on the release button before the delivery member cover, which may cause for example a jamming of the device, and/or in other ways lead to unexpected or unwanted behavior of the device.

### SUMMARY

To at least partially solve the above identified problem with already available such medicament delivery devices, the present disclosure provides an improved medicament delivery device (i.e. “a device”) and an improved rotator for such a device as defined in the accompanying independent claims. Various alternative embodiments of the improved device and rotator are defined in the dependent claims.

According to a first aspect of the present disclosure, a medicament delivery device is provided. The device includes a release button, a delivery member cover, and a rotator.

The device is operable between at least i) a first limiting state, in which the release button is not pushed or partially pushed, where further pushing the release button is possible but not sufficient to cause a triggering of a delivery of a medicament from the device, ii) a second limiting state in which the release button is partially pushed but not sufficiently to cause the triggering, and where further pushing the release button is prevented, and iii) a non-limiting state in which pressing the release button to eventually or directly cause the triggering (of the delivery of the medicament from the device) is possible.

The release button, the delivery member cover and the rotator are so configured and arrange, that: in a first rotational relative alignment of the release button and the rotator, the device is either in the first limiting state or in the second limiting state where, if the device is in the first limiting state, further pushing the release button eventually or directly causes the device to transition to the second limiting state; in a second rotational relative alignment of the release button and the rotator different from the first rotational relative alignment, the device is in the non-limiting state; in the first rotational relative alignment of the release button and the rotator, and with the device in the second limiting state, a transition into the second rotational relative alignment of the release button and the rotator by a torque applied on the rotator, resulting from e.g. pressing on the delivery member cover (towards the rotator), is prevented.

Herein, that pushing the release button “directly” causes something to occur is to be understood as if the release button is already pushed just enough (or at least already in a position) not to cause said something to occur, but that only a minor amount of further pushing is enough to cause said something to occur. That pushing the release button “eventually” causes said something to occur is, consequently, to be understood as if said something will eventually be caused to occur, but that more than just a minor amount of further pushing is needed. For example, “directly causing” may mean that pushing less than one or a few millimeters is enough, such as for example equal to or less than 5 mm or similar, while “eventually causing” may mean that pushing more than a few millimeters, such as for example more than 5 mm or similar, is required. As generally envisaged herein, that any one of the release button and the delivery member cover is “pushed” is to be understood as if they are linearly displaced relative to the rotator. A pressing on the release button and/or the delivery member cover not resulting in such a relative displacement vis-à-vis the rotator is not considered herein as a “pushing”. Phrased differently, it is to be understood that a pressing of something includes exerting a certain force on said something in a certain direction. In addition, pushing of said something includes said something actually also moving in the certain direction, relative to some other object of the device.

In one or more embodiments of the device, the release button and the rotator may each include a first limiting member. The respective first limiting members may be configured and arranged such that, in the first rotational relative alignment of the release button and the rotator, and with the device in the second limiting state, the torque applied on the rotator may eventually or directly cause an edge of one of the first limiting members to press against an edge of the other one of the first limiting members, thereby keeping the device in the second limiting state. Phrased differently, even if torque is still applied on the rotator by e.g. (further) pressing or pushing the delivery member cover, the arrangement of the first limiting members of the release button and the rotator thus keeps the device in the second limiting state by preventing further rotation of the rotator (relative to the release button) in a rotational direction implied by the applied torque.

In one or more embodiments of the device, the first limiting members of the release button and the rotator may be protrusions.

In one or more embodiments of the device, the first limiting member of the release button may be a protrusion and the first limiting member of the rotator may be a corresponding recess, or vice versa, configured such that, in the first rotational relative alignment of the release button and the rotator, and with the device in the second limiting state, the protrusion is within the recess (thereby preventing further rotation of the rotator even if torque is still applied on the rotator by e.g. pressing or pushing the delivery member cover).

In one or more embodiments of the device, the delivery member cover may include a track follower and the rotator may include a track, or vice versa, configured and arranged such that the track follower follows the track, and such that a linear force applied on the delivery member cover when pressing or pushing the delivery member cover may be converted into the torque applied on the rotator.

In one or more embodiments of the device, the release button and the rotator may each include a second limiting member configured and arranged such that, in the first rotational relative alignment of the release button and the rotator, and with the device in the second limiting state, pressing of the release button may cause an edge of one of the second limiting members to press against an edge of the other one of the second limiting members, thereby preventing eventually or directly causing the triggering.

In one or more embodiments of the device, the second limiting member of the rotator may be a cylindrical edge, and the second limiting member of the release button may be a protrusion. For example, it is envisaged that the rotator may be a sleeve having a cylindrical form, and its second limiting member may be the edge of the cylinder shape arranged closest to e.g. the release button. When pressing the release button, its protrusion (i.e. its second limiting member) may press against the cylindrical edge of the rotator, thereby preventing further pushing the release button.

In one or more embodiments of the device, the first limiting member of the rotator may be the second limiting member of the rotator. Phrased differently, both the first limiting member (used to prevent further rotation) and the second limiting member (used to prevent further pushing of the release button) of the rotator may be a same part, for example a same protrusion, a same recess, or similar.

In one or more embodiments of the device, the first limiting member of the release button may be the second limiting member of the release button. Just as for the rotator, both the first limiting member (used to push against the first limiting member of the rotator to prevent rotation of the latter) and the second limiting member (used to push against the second limiting member of the rotator to prevent the release button from being further pushed) of the release button may be a same part, for example a same protrusion, a same recess, or similar.

In one or more embodiments of the device, the first limiting member of one of the release button and the rotator may be a protrusion, and the other one of the release button and the rotator may include a (second) recess. This (second recess) may be configured such that, in the second rotational relative alignment of the release button and the rotator, and with the device in the non-limiting state, the protrusion aligns with the (second) recess such that pressing (i.e. pushing) the release button eventually or directly causes the triggering. Phrased differently, with the (second) recess aligning with the protrusion (i.e. with the first limiting member), the release button may be pushed (i.e. displaced) more, or at all compared to, than if the protrusion and (second) recess were not aligned. In the latter case, the protrusion would instead hit an edge closer to it than e.g. a bottom of the (second) recess.

The present disclosure improves upon existing technology in that the device may only be activated/triggered by a user by first pushing the delivery member cover (such that the device enters the non-limiting state), and only thereafter pushing the release button to trigger the medicament delivery mechanism. This because the release button and the rotator interact such that if the device is in the second limiting state (as a result of the user e.g. mistakenly first at least partially pushing the release button), subsequent pressing of the delivery member cover will not cause the rotator to rotate enough for the device to exit the second limiting state and enter into the non-limiting state. By establishing a sequence dependent activation mechanism, the device as envisaged in the present disclosure therefore has a reduced risk of e.g. jamming if the desired order of pushing the delivery member cover first, and then the release button, is not followed, or for example of accidentally or unexpectedly causing a triggering of the medicament delivery mechanism.

According to a second aspect of the present disclosure, a rotator for a medicament delivery device is provided. It is envisaged that the delivery device includes a release button and a delivery member cover (e.g. as described above and later herein), which are arranged linearly displaceable along a longitudinal axis of the device. The rotator is configured to, when mounted in the device rotatably around the longitudinal axis, mechanically interact with the delivery member cover such that a linear displacement of the delivery member cover relative to the rotator and along the longitudinal axis applies a torque on the rotator around the longitudinal axis. The rotator is configured to, also when mounted in the device rotatably around the longitudinal axis, mechanically interact with the release button such that, in a first rotational relative alignment of the release button and the rotator, the rotator limits a linear displacement of the release button relative to the rotator and along the longitudinal axis, such that pressing the release button towards the rotator directly or eventually causes at least a part of the release button to press against at least a part of the rotator, thereby causing the device to enter into a limiting state wherein a further pushing of the release button to trigger a delivery of a medicament from the device is prevented. The mechanical interaction with the release button is also such that, in a second rotational relative alignment of the release button and the rotator, the device is in a non-limiting state wherein the triggering of the delivery of the medicament from the device by pushing the release button is possible. Further, the mechanical interaction with the release button is such that in the first rotational relative alignment of the release button and the rotator, and with the device in the limiting state, a transition into the second relative alignment of the release button and the rotator by the torque applied to the rotator is prevented.

In one or more embodiments of the rotator, the rotator includes a first limiting member configured to interact with a first limiting member of the release button, such that, in the first rotational relative alignment of the release button and the rotator, and with the device in the limiting state, the torque applied on the rotator eventually or directly causes an edge of the first limiting member of the release button to press against an edge of the first limiting member of the rotator, thereby keeping the device in the limiting state.

In one or more embodiments of the rotator, the first limiting member of the rotator is a protrusion or a recess.

In one or more embodiments of the rotator, the rotator includes a track or a track follower configured to interact with a corresponding track follower or track of the delivery member cover of the device, such that the track or track follower of the rotator follows the track follower or track of the delivery member cover, and such that a linear force applied on the delivery member cover when pressing the delivery member cover is converted into the torque applied on the rotator.

In one or more embodiments of the rotator, the rotator includes a recess configured to, in the second rotational relative alignment of the release button and the rotator, align with said at least a part of the release button such that pushing the release button eventually or directly causes the triggering. Phrased differently, the recess is configured and arranged on the rotator such that in the second relative alignment of the release button and the rotator, the at least a part of the release button is allowed further travelling such that pushing the release button can trigger the delivery of the medicament from the device.

Herein, it is envisaged that the “limiting state” discussed with reference to the rotator of the second aspect may correspond to the “second limiting state” discussed with reference to the device of the first aspect, and vice versa.

Other objects and advantages of the present disclosure will be apparent from the following detailed description, the drawings and the claims. Within the scope of the present disclosure, it is envisaged that all features and advantages described with reference to e.g. medicament delivery device of the first aspect are relevant for, apply to, and may be used in combination with also the those described with reference to the rotator of the second aspect, and vice versa.

### BRIEF DESCRIPTION OF THE DRAWINGS

Exemplifying embodiments will be described below with reference to the accompanying drawings, in which:

FIGS. 1A-1D schematically illustrate various states and relative alignments of components in an embodiment of a medicament delivery device according to the present disclosure, and

FIGS. 1E-F schematically illustrate various other embodiments of medicament delivery devices according to the present disclosure.

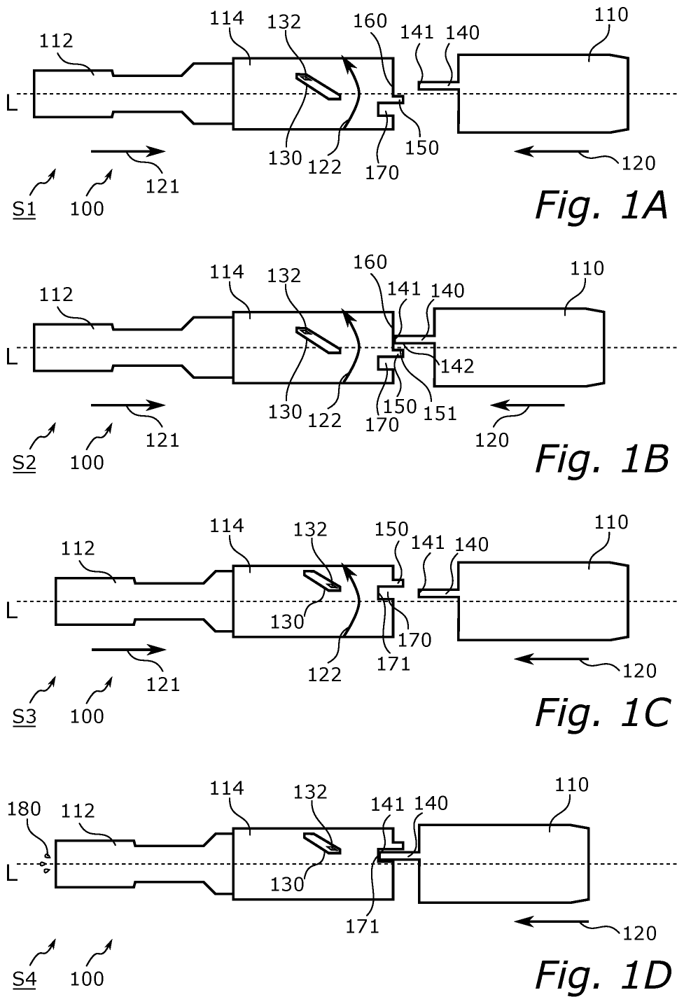

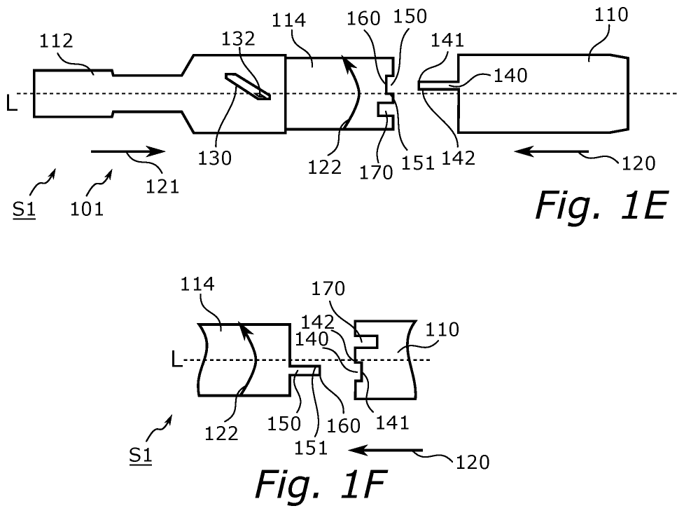

Although described as illustration a device, FIGS. 1A-1F also serves to illustrate various embodiments of a rotator according to the present disclosure, and its interaction with the device as a whole.

In the drawings, like reference numerals will be used for like elements unless stated otherwise. Unless explicitly stated to the contrary, the drawings show only such elements that are necessary to illustrate the example embodiments, while other elements, in the interest of clarity, may be omitted or merely suggested. As illustrated in the Figures, the (absolute or relative) sizes of elements and regions may be exaggerated or understated vis-à-vis their true values for illustrative purposes and, thus, are provided to illustrate the general structures of the embodiments.

### DETAILED DESCRIPTION

Exemplifying embodiments of a medicament delivery device (and a rotator for such a device) according to the present disclosure will now be described more fully hereinafter with reference to the accompanying drawings. The drawings show currently preferred embodiments, but the invention may, however, be embodied in many different forms and should not be construed as limited to the embodiments set forth herein; rather, these embodiments are provided for thoroughness and completeness, and fully convey the scope of the present disclosure to the skilled person.

The various components of a device (including a rotator) as envisaged herein, as well as the various states in which the device (and rotator) can be operated, will now be described with reference to FIGS. 1A-1F.

FIG. 1A schematically illustrates a device 100 which is currently in its first limiting state S1. The device 100 includes a release button 110 which is arranged such that it may be pressed/displaced linearly in a direction 120 along a longitudinal axis L of the device 100.

The release button 110 may for example be spring loaded by using a spring (not shown) which provides, when the release button 110 is pressed in the direction 120, a counteracting force in a direction opposite to the direction 120. The spring constant of the spring may be selected low enough such that e.g. a user's thumb pressing on the release button 110 is strong enough to overcome the counteracting force provided by such a spring, but high enough such that e.g. shaking of the device 100 does not cause any accidental pushing of the release button 110.

The device 100 further includes a delivery member cover 112. The delivery member cover 112 may be hollow and shaped as for example a sleeve/cylinder, and functions to cover a delivery member (such as e.g. a needle, not shown) from being accessible by a user in the state S1 of the device 100. The delivery member cover 112 is, just like the release button 110, also arranged in the device 100 such that it may be pressed and thereby displaced linearly along the longitudinal axis L, but in a direction 121 opposite that of the direction 120. The delivery member cover 112 may also be spring loaded by a spring (also not shown), which spring may provide, when the delivery member cover is pressed in the direction 121, a counteracting force in a direction opposite to the direction 121. The spring constant for this spring may be selected low enough such that e.g. a hand of the user is strong enough to overcome the counteracting force when pressing the delivery member cover and the device against e.g. the user's skin, but high enough such that the delivery member cover is not accidentally pressed by e.g. shaking the device 100 or letting the device 100 just rest against the user's skin.

The device 100 further includes a rotator 114. The rotator 114 is arranged within the device 100 such that it may be rotated around the longitudinal axis L, and rotated relative to release button 110 and the delivery member cover 112. The rotator 114 may be hollow and shaped for example like a sleeve/cylinder. The rotator 114 has a track 130 which is arranged such that a track follower 132 of the delivery member cover 112 is within the track 130. This arrangement of track 130 and track follower 132 allows a linear displacement of the delivery member cover 112 in the direction 121 to cause a torque 122 being applied to the rotator 114. If the rotator 114 is not locked by other means, the applied torque 122 causes a rotation of the rotator 114 around the longitudinal axis L of the device. Phrased differently, the arrangement of the track 130 of the rotator 114 and the track follower 132 of the delivery member cover 112 allows for a linear displacement (i.e. a pushing) of the delivery member cover 112 to be converted into a rotational displacement (i.e. a rotation) of the rotator 114.

Herein, that a component is “displaced linearly” is to be understood as said component being displaced linearly relative to one or more other components of the device 100, along the longitudinal axis L, and does mean e.g. any linear displacement of e.g. the device 100 as a whole. Pushing of the release button 110 may for example mean that the release button 110 moves towards the rotator 114 (and also towards the delivery member cover 112) along the longitudinal axis L. Pushing of the delivery member cover 112 may for example mean that the delivery member cover 112 moves towards the rotator 114 (and also towards the release button 110) along the longitudinal axis L.

The release button 110 is further configured to, if pressed (i.e. linearly displaced) sufficiently in the direction 120, trigger a medicament delivery mechanism (not shown) within the device 100. When triggered, the medicament delivery mechanism can cause an amount of medicament stored within the device (in for example an ampulla, also not shown) to be ejected through the delivery member (e.g. through a needle). However, in the state S1 of the device as illustrated in FIG. 1A, the release button 110 is prevented from being pressed sufficiently to cause such triggering. This is obtained as follows:

The release button 110 further includes a first limiting member in form of a protrusion 140 which extends out towards the rotator 114 from a side/surface of the release button 110 arranged towards the rotator 114. The rotator has an edge 160 facing towards the protrusion 140 of the release button 110. The edge 160 includes a recess 170 therein, and the rotator 114 also has a first limiting member in the form of a protrusion 150 extending out from the edge 160 towards the release button 110.

In the state S1 of the device 100, a rotational relative alignment of the release button 110 and the rotator 114 is such that the protrusion 140 of the release button 110 does not align with the recess 170 of the rotator 114. Consequently, the release button 110 may be pressed in the direction 120, but only until an edge 141 of the protrusion 140 abuts/presses against the edge 160 of the rotator. The relative displacement of the release button 110 so achieved is not sufficient to cause the triggering of the medicament delivery mechanism. Instead, pushing or pressing the release button 110 such that the edge 141 of the protrusion 140 abuts/presses against the edge 160 will bring the device 100 into its second limiting state S2, which will be described below with reference to FIG. 1B.

FIG. 1B schematically illustrates the same device 100 but in its second limiting state S2. In the second limiting state S2, the release button 110 is pressed such that the edge 141 of the protrusion 140 abuts/presses against the edge 160 of the rotator 114, thereby preventing any further pushing of the release button 110 in the direction 120 relative to the rotator 114. Further pushing the release button 110 to eventually or directly cause the triggering of the medicament delivery mechanism is therefore not possible.

Pressing or pushing on the delivery member cover 112 in the direction 121 may however apply (or continue to apply) the torque 122 to the rotator 114. If the pressing of (i.e. the force applied to) the delivery member cover 112 is large enough, it is envisaged that the generated torque 122 may be sufficiently large to overcome a friction between the edge 141 of the protrusion 140 and the edge 160 of the rotator 114, and thereby cause the rotator 114 to start rotating relative the release button 110. To avoid such torque from transitioning the device 100 out of the second limiting state S2, the protrusion 140 of the release button 110 and the protrusion 150 of the rotator 114 (i.e. the first limiting members) are configured to interact as follows:

When applying the torque 122 to the rotator 114, a rotation of the rotator 114 may eventually cause a lateral edge 142 of the protrusion 140 to abut/press against a lateral edge 151 of the protrusion 150. When this happens, further rotation of the rotator 114 in the direction implied by the torque 122 will be prevented, such that the rotator 114 may not rotate sufficiently to e.g. align its recess 170 with the protrusion 140 of the release button 110. Phrased differently, in the second limiting state S2 of the device, the first limiting members (i.e. the protrusions 140 and 150) of the release button 110 and rotator 114 are configured to prevent the torque 122 from transitioning the device 100 out of the second limiting state S2 and into e.g. a non-limiting state (as will be described below) where pushing the release button 110 to cause the triggering of the medicament delivery mechanism is possible. Herein, it is to be understood that any rotational relative alignment of the release button 110 and the rotator 114 wherein such triggering is not possible (due to the interaction of the protrusion 140 with one or both of the edge 151 and the protrusion 150 of the rotator 114) can be referred to as a “first rotational relative alignment” of the release button 110 and the rotator 114.

FIG. 1C schematically illustrates another, second rotational relative alignment of the release button 110 and rotator 114, when the device 100 itself is in what is referred to as a non-limiting state S3. Here, the release button 110 is not sufficiently pressed for the protrusion 140 to interact with the rotator 112 to prevent rotation of the latter. The delivery member cover 112 has been pressed in the direction 121, and the torque 122 so generated (by the interaction of the track 130 of the rotator 114 and the track follower 132 of the delivery member cover 112) has caused the rotator 114 to rotate around the longitudinal axis L of the device 100 such that the recess 170 is aligned with the protrusion 140. Consequently, in the state S3 of the device 100, the release button 110 can be pressed in the direction 120 until the edge 141 of the protrusion 140 abuts/presses against an edge/bottom 171 of the recess 170. It is envisaged that the recess 170 is deep enough such that a maximum displacement of the release button 110 relative the rotator 114 is sufficiently large for the release button 110 to trigger the medicament delivery mechanism to eject the medicament through the delivery member.

FIG. 1D schematically illustrates the device in such a triggered state S4, wherein the release button 110 has been sufficiently pressed in the direction 120 to trigger a delivery of a medicament 180 from the device 100. In the triggered state S4, the edge 141 of the protrusion 140 abuts/presses against the edge/bottom 171 of the recess 170, and even further pushing of the release button is not possible. It may also be envisaged that there are one or more other components which blocks linear movement of the release button 110 relative to the rotator 114 before the former comes into contact with the latter, but that such a blocking occurs only after the release button 110 has been sufficiently displaced relative to the rotator 114 to trigger the medicament delivery mechanism.

In summary of FIGS. 1A-1D, it can be concluded that the various components of the device 100 are configured to interact such that if the release button 110 is pressed before the delivery member cover 112, and such that the device 100 thereby have entered the second limiting state S2, triggering of the medicament delivery mechanism cannot be accidentally performed by pushing/further pressing on the delivery member cover 112. This is obtained by the arrangement and interaction of the first limiting members (i.e. the protrusions 140 and 150), such that sufficient rotation of the rotator 114 to align its recess 170 with the protrusion 140 of the release button 110 is not possible. This provides a safer medicament delivery device, wherein the order of pushing the release button 110 and the delivery member cover 112 is important, and wherein the risk of the user jamming the device by pushing/pressing the release button 110 first is reduced. In such a situation, a triggering of the medicament delivery mechanism is only possible if first letting go of the release button 110, and then pushing the delivery member cover 112 before the release button 110 is pressed again.

FIG. 1E schematically illustrates another envisaged embodiment of a medicament delivery device 101 in the second limiting state S1. Compared with the device 100 described above with reference to FIGS. 1A-1D, in the device 101 the track follower is instead part of the rotator 114, while the track 130 is part of the delivery member cover 112. Also, the first limiting member of the rotator 114 is not a protrusion, but rather a recess 150 arranged in the rotator 114 on its side facing the release button 110. The edge 160 is a bottom edge of the recess 150, and an edge 151 of the recess 150 has the same function as the edge 151 of the protrusion 150 of the device 100 described above with reference to FIGS. 1A-1D. Else, the interactions of the various components follow those of the device 100, and the device 101 is only one example of how the first limiting members of the release button 110 and the rotator 114 may be configured to interact as desired while their exact forms and/or locations may vary from device to device.

A further example of such variations is illustrated in FIG. 1F, which schematically illustrates another configuration of the release button 110 and the rotator 114 in a device being in the first limiting state S1. Here, in contrast to the device 101 described with reference to FIG. 1E, the first limiting member of the rotator 114 is a protrusion 150, while the first limiting member of the release button 110 is instead a recess 170. In addition, the recess 170 which in e.g. devices 100 and 101 was part of the rotator 114 is instead part of the release button 110. Phrased differently, the configuration shown in FIG. 1F may be though of as a mirror of that shown in FIG. 1E, as the protrusion 140 and the recesses 150/170 have all been moved to the other one of the release button 110 and the rotator 114. The envisaged interactions of the various components however cause the same functionality as described with reference to e.g. FIGS. 1A-1D.

In other envisaged embodiments, the release button 110 may for example be shaped like a sleeve/partly hollow cylinder and fit around the outside of the rotator 114. To prevent the further pushing of the release button 110 needed to trigger the medicament delivery mechanism, the rotator 114 may then for example include a rim on its outside against which an edge of the release button 110 may press/abut when the device is in the second limiting state. Cutouts may be provided in both e.g. the rim and the edge of the release button 110 and arranged such that, in the second rotational relative alignment of the release button 110 and the rotator 114, further movement (i.e. beyond the rim) of the release button is possible such that the release button may be pressed/pushed to directly or eventually cause the triggering of the medicament delivery. Other means of limiting the travel of the release button 110 (to prevent its further pushing) in the first rotational relative alignment but not the second rotational relative alignment are of course also possible.

Although not explicitly disclosed in any drawings herein, it is envisaged that the medicament delivery device may also include additional components, including e.g. a protective cap for the delivery member, the delivery member itself in form of e.g. a needle and/or syringe, an ampulla, syringe, or cartridge for storing the dose of medicament, one or more guiding rods and or other structures for the arrangement of the delivery member cover, the release button and the rotator as desired, a plunger rod and preloaded spring for, as part of the medicament delivery mechanism, force the medicament out of the ampulla/syringe when triggered, etc. For example, the medicament delivery mechanism may for example include a preloaded spring which, when release, forces a plunger rod to both puncture the injection site using the delivery member, and to thereafter force the dose of medicament out through the delivery member and into the body of the user. The release of the preloaded spring may be caused by a part of the release button, when the button is sufficiently pushed to cause such triggering, releases a holding mechanism for the spring, such that the spring may be free to exert a spring force on the plunger rod. For example, the spring may be held in a compressed/preloaded state by one or more gripping tabs. The release button may include a member which, when the release button is sufficiently pushed, interacts with the gripping tabs causing them to lose their grip of the spring. Other variants are of course also envisaged, as long as their overall functionality is the same as described herein.

Medicament delivery devices as envisaged herein may for example be autoinjectors containing insulin, allergy medicaments, epinephrine, migraine medicaments, atropine, or any other medicament/drug for which the use of a medicament delivery device as described herein is suitable. The device can be of a single-use type (i.e. disposable), or the device may be of a type which can be reloaded after use and reused one or more times. Delivery members may include needles, syringes, syrettes, or other suitable structures.

In the present disclosure, the term “longitudinal axis” of the device refers to an axis extending from a proximal end of the device to a distal end of the device, typically a central axis along the device in the direction of longest extension of the device. Likewise, “rotation”, “rotatably” an “rotational” refer to rotation relative to the longitudinal axis. The term “distal end” refers to the part/end of the device, or the parts/ends of the members thereof, which under use of the device is/are located furthest away from the dose delivery/injection site. Correspondingly, the term “proximal end” refers to the part/end of the device, or the parts/ends of the members thereof, which under use of the device is/are located closest to the dose delivery/injection site.

Although features and elements may be described above in particular combinations, each feature or element may be used alone without the other features and elements or in various combinations with or without other features and elements. Additionally, variations to the disclosed embodiments may be understood and effected by the skilled person in practicing the claimed invention, from a study of the drawings, the disclosure, and the appended claims.

In the claims, the words “comprising” and “including” does not exclude other elements, and the indefinite article “a” or “an” does not exclude a plurality. The mere fact that certain features are recited in mutually different dependent claims does not indicate that a combination of these features cannot be used to advantage.

1. A medicament delivery device (100), comprising:
a release button (110);
a delivery member cover (112), and
a rotator (114),
wherein the device is operable between at least i) a first limiting state (S1) in which the release button is not pushed or partially pushed, where further pushing (120) the release button is possible but not sufficient to cause a triggering of a delivery of a medicament from the device, ii) a second limiting state (S2) in which the release button is partially pushed but not sufficiently to cause the triggering, and where further pushing (120) the release button is prevented, and iii) a non-limiting state (S3) in which pressing the release button to eventually or directly cause the triggering is possible, and
wherein the release button, the delivery member cover and the rotator are so configured and arranged, that:
in a first rotational relative alignment of the release button and the rotator, the device is either in the first limiting state or in the second limiting state where, if the device is in the first limiting state, further pressing the release button eventually or directly causes the device to transition to the second limiting state;
in a second rotational relative alignment of the release button and the rotator different from the first rotational relative alignment, the device is in the non-limiting state;
in the first rotational relative alignment of the release button and the rotator, and with the device in the second limiting state, a transition into the second rotational relative alignment of the release button and the rotator by a torque (122) applied on the rotator, is prevented.

2. The device according to claim 1, wherein the release button and the rotator each includes a first limiting member (140, 150) configured and arranged such that:
in the first rotational relative alignment of the release button and the rotator, and with the device in the second limiting state, the torque applied on the rotator eventually or directly causes an edge (141) of one of the first limiting members to press against an edge (151) of the other one of the first limiting members, thereby keeping the device in the second limiting state.

3. The device according to claim 2, wherein the first limiting members (140,150) of the release button and the rotator are protrusions.

4. The device according to claim 2, wherein the first limiting member of the release button is a protrusion and the first limiting member of the rotator is a corresponding recess, or vice versa, configured and arranged such that:
in the first rotational relative alignment of the release button and the rotator, and with the device in the second limiting state, the protrusion is within the recess.

5. The device according to claim 1, wherein the delivery member cover includes a track follower (132) and the rotator includes a track (130), or vice versa, configured and arranged such that:
the track follower follows the track, and such that a linear force applied on the delivery member cover when pressing (121) the delivery member cover is converted into the torque (122) applied on the rotator.

6. The device according to claim 1, wherein the release button and the rotator each includes a second limiting member configured and arranged such that:
in the first rotational relative alignment of the release button and the rotator, and with the device in the second limiting state, pressing of the release button causes an edge of one of the second limiting members to press against an edge of the other one of the second limiting members, thereby preventing eventually or directly causing the triggering.

7. The device according to claim 6, wherein the second limiting member of the rotator is a cylindrical edge, and the second limiting member of the release button is a protrusion.

8. The device according to claim 2, wherein the first limiting member of the rotator is the second limiting member of the rotator.

9. The device according to claim 2, wherein the first limiting member of the release button is the second limiting member of the release button.

10. The device according to claim 1, wherein the first limiting member of one of the release button and the rotator is a protrusion, and wherein the other one of the release button and the rotator includes a second recess, configured and arranged such that:
in the second rotational relative alignment of the release button and the rotator, and with the device in the non-limiting state, the protrusion aligns with the second recess such that pressing the release button eventually or directly causes the triggering.

11. A rotator (114) for a medicament delivery device (100), wherein the medicament delivery device (100) includes a release button (110) and a delivery member cover (112) arranged linearly displaceable along a longitudinal axis (L) of the device, wherein the rotator is configured to, when mounted in the device rotatably around the longitudinal axis (L):
mechanically interact with the delivery member cover such that a linear displacement of the delivery member cover relative to the rotator and along the longitudinal axis applies a torque (122) on the rotator around the longitudinal axis, and
mechanically interact with the release button such that:
in a first rotational relative alignment of the release button and the rotator, the rotator limits a linear displacement of the release button relative to the rotator and along the longitudinal axis, such that pressing the release button towards the rotator directly or eventually causes at least a part of the release button to press against at least a part of the rotator, thereby causing the device to enter into a limiting state wherein a further pushing of the release button to trigger a delivery of a medicament from the device is prevented;
in a second rotational relative alignment of the release button and the rotator, the device is in a non-limiting state wherein the triggering of the delivery of the medicament from the device by pressing the release button is possible, and
in the first rotational relative alignment of the release button and the rotator, and with the device in the limiting state, a transition into the second rotational relative alignment of the release button and the rotator by the torque (122) applied on the rotator is prevented.

12. The rotator according to claim 11, comprising a first limiting member (150) configured to interact with a first limiting member (140) of the release button such that:
in the first rotational relative alignment of the release button and the rotator, and with the device in the limiting state, the torque applied on the rotator eventually or directly causes an edge (141) of the first limiting member of the release button to press against an edge (151) of the first limiting member of the rotator, thereby keeping the device in the limiting state.

13. The rotator according to claim 12, wherein the first limiting member of the rotator is a protrusion or a recess.

14. The rotator according to claim 11, comprising a track (130) or a track follower (132) configured to interact with a corresponding track follower or track of the delivery member cover of the device, such that the track or track follower of the rotator follows the track follower or track of the delivery member cover, and such that a linear force applied on the delivery member cover when pressing (121) the delivery member cover is converted into the torque (122) applied on the rotator.

15. The rotator according to claim 11, comprising a recess (170) configured to, in the second rotational relative alignment of the release button and the rotator, align with said at least a part of the release button such that pressing the release button eventually or directly causes the triggering.
